(19) United States
(12) Patent Application Publication
(10) Pub. No.: US 2006/0224175 A1
(43) Pub. Date: Oct. 5, 2006

(54) Methods and apparatuses for disposition of a medical device onto an elongate medical device

(76) Inventor: Anthony C. Vrba, Maple Grove, MN (US)

Correspondence Address: CROMPTON, SEAGER & TUFTE, LLC, 1221 NICOLLET AVENUE, SUITE 800, MINNEAPOLIS, MN, 55403-2420, US

(21) Appl. No.: 11/092,082

(22) Filed: Mar. 29, 2005

(51) Int. Cl. A61M 29/00

(52) U.S. Cl. 606/200

(57) ABSTRACT

A percutaneous medical device that may include a stopper having a first end, a second end and a lumen therebetween, the stopper having first state where the lumen has a first cross-sectional area and a second state where the lumen has a second cross-sectional area less than the first cross-sectional area, the stopper having a slit between the first end and the second end when in the first state, the slit defining a gap, the stopper when in the second state having no bias to move to the first state at body temperature, the stopper having a distance of less than 3 cm between the first end and the second end and apparatus and methods of use pertaining thereto.

### FIELD OF THE INVENTION

The invention pertains to methods and apparatuses that permit a medical practitioner such as a doctor to add functionality such as distal protection to medical devices such as guidewires.

### BACKGROUND

Guidewires are one of the most common devices used in intravascular procedures. They are designed to be easy to navigate to the treatment site and they permit other therapeutic devices to be advanced easily to the treatment. Guidewires with a distal protection element such as a filter or balloon are becoming more common. A distal protection element provides one common means of controlling any emboli that may be created by a procedure. Guidewires may be highly specialized to provide quicker or easier access to particular intravascular sites and it may be difficult or expensive for a hospital or clinic to keep in stock all the guidewires a practitioner may desire to use. This problem (i.e. keeping a wide stock of guidewires on hand) is compounded with the introduction of new features such as distal protection to guidewires.

### SUMMARY

One embodiment of the invention pertains to a method of converting a non-distal protection guidewire into a distal protection guidewire. A desired guidewire is selected by the medical practitioner. A stopper is affixed to the guidewire at a desired location and a desired distal protection device is loaded onto the guidewire to create a distal protection guidewire. The stopper may be distal to the distal protection device or may be proximal. In one embodiment, the distal protection device is trapped between a distal stopper and a proximal stopper. In another embodiment, the stopper is integral with the distal protection device.

Another embodiment of the invention pertains to a stopper for the conversion of a guidewire. The stopper has a first state where it has a larger lumen enabling it to be slid to a desired location on a guidewire and a second state where it has a smaller lumen and may be affixed to the guidewire. The stopper may be affixed by crimping or by adhesive. Another embodiment is a stopper made from a shape memory material that is affixed to a guidewire when it approaches body temperature. Another embodiment is made from a spring material that is biased to be in the second state.

### BRIEF DESCRIPTION OF DRAWINGS

The invention may be more completely understood in consideration of the following detailed description of various embodiments of the invention in connection with the accompanying drawings in which:

FIG. 1 is a partial plan view of a guidewire in a vascular lumen, the guidewire having a distal protection device and a stopper disposed thereon;

FIG. 2A is an end view of a stopper in a first state;

FIG. 2B is an end view of a stopper in a second state;

FIG. 3 is an orthogonal view of a stopper;

FIG. 4 is an orthogonal view of a stopper;

FIG. 5 is a partial plan view of a guidewire in a vascular lumen, the guidewire having a distal protection device and two stoppers disposed thereon; and

FIG. 6 is a partial plan view of a guidewire in a vascular lumen, the guidewire having a distal protection device having an integral stopper disposed thereon.

### DETAILED DESCRIPTION OF SELECT EMBODIMENTS

Reference is now made to the figures, in which like element numbers refer to like elements throughout.

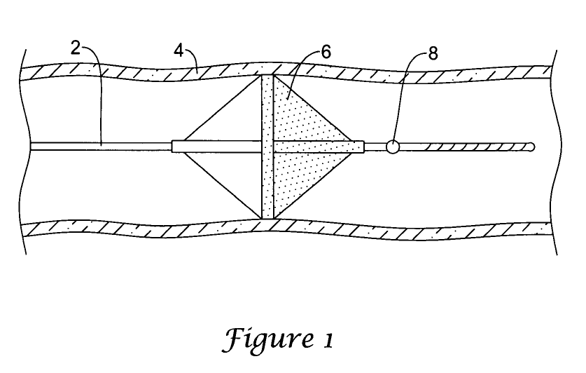

FIG. 1 is a plan view of an example guidewire 2 disposed in a vascular lumen defined by a vascular wall 4. Slideably disposed on guidewire 2 is a distal protection device 6. A stopper 8 is disposed distal of distal protection device 6 on guidewire 2. Stopper 8 prevents distal movement of distal protection device past the stopper. Thus a guidewire can be made into a distal protection guidewire. The term guidewire is herein merely a proxy for any elongate medical device. Thus a catheter, wire, or other elongate medical device may be suitable. Likewise, the term distal protection device is merely a proxy for any suitable device. The device merely needs a lumen through which the guidewire may be inserted.

This combination can be made by a medical practitioner at a hospital or clinic shortly prior to the therapeutic procedure in which the distal protection guidewire is used. Thus a distal protection guidewire can be customized by selecting a preferred guidewire, a preferred distal protection device and a preferred arrangement of the components (i.e. where the distal protection device is placed on the guidewire). Selective use of one or more stoppers 8 is used to make the conversion.

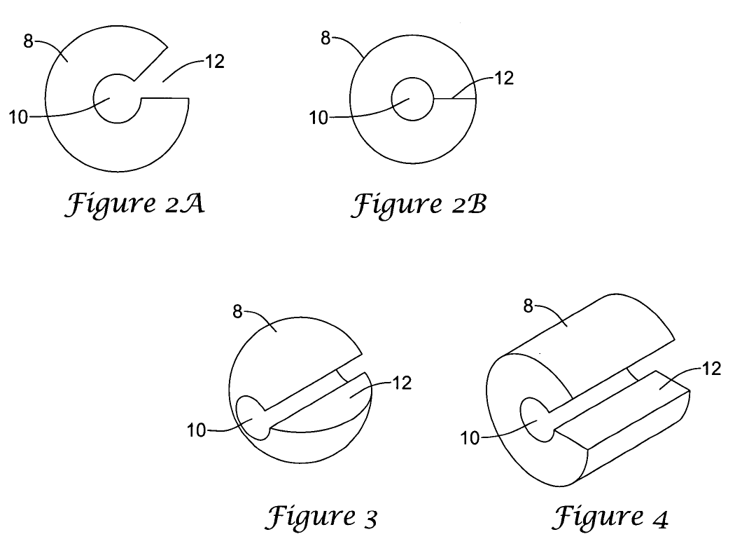

A stopper 8 has a longitudinal lumen 10 as can be seen in FIG. 2A, which is an end view of a stopper 8. In a first state, longitudinal lumen 10, which extends from a first end to a second end, has a first cross-sectional area that is sufficient to permit the stopper to slide over a selected guidewire to a selected location. In the first state, slit 12 defines a gap. Upon installation, the stopper is moved to a second state, where lumen 10 has a second cross-sectional area smaller than the first cross-sectional area of the first state. Slit 12 may be completely closed, as seen in FIG. 2B, or may merely define a smaller gap than in the first state. In other words, in the second state, the stopper may completely encircle the guidewire or may only partially do so. The stopper may have a spherical profile as shown in FIG. 3 or a cylindrical profile as shown in FIG. 4. The stopper may have other suitable profiles as well. Any profile which prevents a distal protection device from passing over it may be suitable. For example, the stopper may have a flared first end that tapers to a narrower second end. In the second state, stopper 8 fits tightly over a guidewire, fixing it to the guidewire. In some embodiments, the stopper has an interference fit with the guidewire and lumen 10 is completely occluded by the guidewire. In some embodiments, the inner surface of the stopper, which defines lumen 10, has a texture such as a plurality of ribs or wales that help to fix the stopper to the guidewire. In other embodiments, a pressure or heat sensitive adhesive may be used to help fix the stopper to the guidewire.

In one embodiment, the stopper is made from a plastically deformable material such as gold, silver, tin, or alloys thereof. Other such materials may be suitable. The term “plastically deformable” herein means that the stopper is made of such a material that, in moving from the first state to the second state, it will undergo plastic deformation. Such a stopper may be affixed to a guidewire by crimping. A crimping tool such as a pliers may be provided. The crimping tool may have jaws to receive the stopper and the jaws may have a pair of surfaces that have profiles that are inverse of the profile of the stopper. For example, if the stopper has a spherical profile, the jaws may include a pair of spherical indents sized and shaped to receive the stopper. The crimping tool may also include a stop to prevent over-crimping of the stopper. This stop may be a pair of opposing surfaces on the jaws or handles or may be any suitable stop. In some embodiments, a stopper may be packaged preloaded in the jaws of a suitable crimping tool.

In another embodiment, the stopper is a made from a shape memory material such as a nickel-titanium alloy. Other shape memory materials may also be suitable. The stopper moves to the first state when brought to body temperature. Body temperature herein means the normal range of temperatures found in the human body. The stopper moves to the second state when heated up from body temperature. In an alternative embodiment, the stopper may move to the second state when cooled down from body temperature. In some embodiments, the temperature at which the stopper moves to the second state is higher than body temperature, 150 degrees Fahrenheit for example, but not so hot as to melt the polymers typically used in many guidewires. In one example installation, this stopper is heated to an installation temperature where it is in the first state and slid onto a guidewire, where it is allowed to cool down to the second state. The cooling may be hastened if desired by immersing the stopper in a sterile fluid or pouring a sterile fluid over the stopper.

In another example embodiment, the stopper is made from a spring material such as a spring steel that is biased towards the second state. The stopper is expanded to the first state, slid to a desired location on a selected guidewire and released to snap back to the second state. In one embodiment, the stopper is packaged in the second state, with a spacer holding slit 12 open. The spacer may have a handle thereon with which the stopper/spacer combination may be easily manipulated and with which the spacer can be easily removed from the stopper.

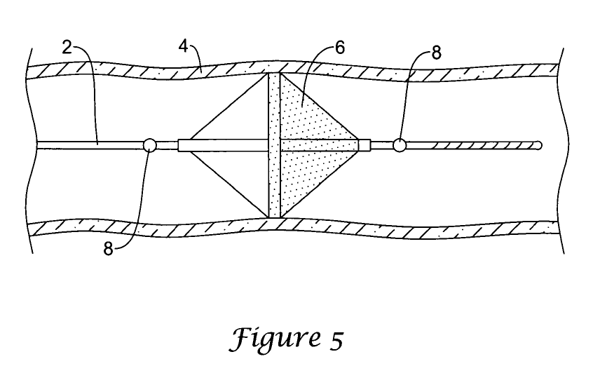

FIG. 5 is a plan view of a guidewire 2 in a vascular lumen defined by a vascular wall 4. Guidewire 2 has had two stoppers 8 attached to it, trapping a distal protection device 6 therebetween. Distal protection device 6 can rotate freely with respect to the guidewire but has limited proximal and distal movement.

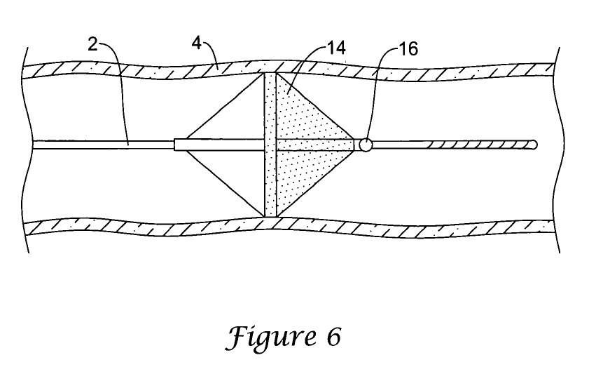

FIG. 6 is a plan view of a guidewire 2 in a vascular lumen defined by a vascular wall 4. A distal protection device 14 having an integrated stopper 16 is disposed on guidewire 2. Integrated stopper 16 may affix to guidewire 2 in substantially the same way as any of the stoppers 8. In some embodiments, the outer profile of stopper 16 may be significantly lower. Stopper 16 is depicted at the distal end of distal protection device 14 although in some embodiments the stopper may be located elsewhere on the distal protection device, such as in the middle or at the proximal end. Some embodiments may include more than one stopper 16.

In one example use, a guidewire is modified by a medical practitioner as described above and as shown in one of FIG. 4, 5, or 6. The distal protection device may be confined in a catheter in a compact configuration. The guidewire and catheter are introduced concurrently percutaneously to a desired location such as the lumen of a blood vessel distal a treatment site. The catheter may then be removed to release the distal protection device. Other therapeutic devices such as an atherectomy device, stent deployment balloon or angioplasty balloon may then be introduced over the guidewire, if desired.

In another example use, a stopper 8 is affixed to a guidewire. The guidewire is then introduced percutaneously. A distal protection device is then introduced over the guidewire. The stopper 8 ensures the distal protection device won't slide off the distal end of the guidewire.

In another example use, a stopper 8 is affixed to a guidewire. An implantable filter such as a vena cava filter is loaded distal the stopper and is confined in a compact position by a catheter. The filter is introduced to a desired location and the guidewire may be used to push the filter distally from the end of the catheter or may be used to keep the filter from moving proximally as the catheter is withdrawn from over the filter. The catheter and guidewire are then withdrawn, leaving the filter installed.

To provide a medical practitioner with flexibility, stoppers 8 may be offered in various sizes to fit on guidewires of various outer diameters. Each stopper may be packaged in a separate sterile package. Installation tools may be manufactured inexpensively from a small number of parts such as molded plastic parts and each stopper may be packaged preloaded in an appropriate installation tool in a sterile package. The stoppers may also be packaged as a set with one or two stoppers packaged with an appropriate distal protection device. The packaging may include a spacer passing through the lumen of the stopper, which may provide easier handling of the stopper and may keep the stopper in the first state. The stopper may be easily slid off the spacer

Numerous advantages of the invention covered by this document have been set forth in the foregoing description. It will be understood, however, that this disclosure is, in many respects, only illustrative. Changes may be made in details, particularly in matters of shape, size, and arrangement of parts without exceeding the scope of the invention. None of the description in the present application should be read as implying that any particular element, step, or function is an essential element which must be included in the claim scope. Moreover, none of these claims are intended to invoke 35 U.S.C. § 112, ¶ 6 unless the exact words “means for” are followed by a participle. The invention's scope is, of course, defined in the language in which the appended claims are expressed.

What is claimed is:

1. A kit for the conversion of a guidewire into a distal protection guidewire, comprising:
a distal protection member having a lumen therethrough for receiving any guidewire having a outer diameter of a certain size or less; and
a stopper fixable to a guidewire, the stopper having a lumen extending from a proximal end of the stopper to a distal end of the stopper.

2. The kit of claim 1, wherein the lumen of the stopper is substantially empty.

3. The kit of claim 1, wherein the stopper has a first state where the lumen has a first cross-sectional area and a second state where the lumen has a second cross-sectional area less than the first cross-sectional area.

4. The kit of claim 3, where the stopper has an arcuate shape in the first state and a closed loop shape in the second state.

5. The kit of claim 1, further comprising a second stopper fixable to a guidewire, the second stop having the same size as the first stopper.

6. The kit of claim 1, further comprising a second stopper fixable to a guidewire, the second stopper being fixable to guidewire of larger cross-sectional area than the first stopper.

7. The kit of claim 1, wherein the stopper is plastically deformable from the first state to the second state.

8. The kit of claim 1, further comprising a tool for fixing the stopper to a guidewire.

9. The kit of claim 8, wherein the tool has a pair of jaws having opposing surfaces whose profiles correspond to the profile of the outer surface of the stopper.

10. The kit of claim 8, wherein the tool has a stopper for preventing the jaws from closing beyond a certain point.

11. The kit of claim 10, wherein the stopper is a pair of opposing surfaces on the jaws.

12. The kit of claim 1, wherein the stopper is attached to the distal protection member

13. A method of converting a guidewire into a distal protection guidewire, comprising the steps of:
providing a distal protection member having a distal end a proximal end and a lumen therebetween, the distal protection member having a first compact state and a second expanded state for trapping emboli; and
providing a first stopper defining an inner cross-sectional area, the stopper having a first state where the inner cross-sectional area has a first magnitude and a second state where the inner cross-sectional area has a second magnitude less that the first magnitude, the stopper having an outer profile in the second state such that the distal protection member cannot pass over it.

14. The method of claim 13, wherein the stopper is compressible from the first state to the second state.

15. The method of claim 13, further comprising the step of providing a tool for fixing the stopper to a guidewire.

16. The method of claim 13, further comprising the step of providing a second stopper, the second stopper having a first state where the inner cross-sectional area has a first magnitude and a second state where the inner cross-sectional area has a second magnitude less that the first magnitude, the second stopper having an outer profile in the second state such that the distal protection member cannot pass over it.

17. The method of claim 13, wherein the step of providing the first stopper includes the step of providing the first stopper in the first state to a medical facility that performs therapeutic treatments of patients.

18. A method of converting a guidewire into a distal protection guidewire, comprising the steps of:
providing a first stopper in a first state to a medical facility that performs therapeutic treatments of patients, the stopper defining a lumen having an inner cross-sectional area, the first state being where the inner cross-sectional area has a first magnitude and the stopper having a second state where the inner cross-sectional area has a second magnitude less that the first magnitude, the stopper having an outer profile in the second state such that the distal protection member cannot pass over it.

19. A percutaneous medical device, comprising
a stopper having a first end, a second end and a lumen therebetween,
the stopper having first state where the lumen has a first cross-sectional area and a second state where the lumen has a second cross-sectional area less than the first cross-sectional area,
the stopper having a slit between the first end and the second end when in the first state, the slit defining a gap,
the stopper when in the second state having no bias to move to the first state at body temperature,
the stopper having a distance of less than 3 cm between the first end and the second end; and
no member having a length of greater than 10 cm.

20. The device of claim 19, wherein the stopper has an inner surface having a texture.

21. The device of claim 20, wherein the texture is a plurality of ribs disposed on the inner surface.

22. The device of claim 19, wherein the stopper has a cylindrical profile in the second state.

23. The device of claim 19, wherein the stopper has a spherical profile in the second state.

24. The device of claim 19, wherein the stopper Is plastically deformable.

25. The device of claim 24, wherein the stopper includes a material selected from the group of tin, silver and gold.

26. The device of claim 25, wherein the stopper comprises a shape memory material that biases the stopper to the second state at body temperature.

27. The device of claim 26, wherein the stopper is biased to the first state at a temperature higher than body temperature.

28. The device of claim 19, wherein the stopper Is made of a material that is elastically deformable between the first state and the second state.

29. The device of claim 19, further comprising a sterile package surrounding the stopper.

30. The device of claim 29, wherein the lumen is empty.

31. The device of claim 29, further comprising a spacer removably disposed in the lumen.

32. The device of claim 19, wherein the stopper is affixable to a guidewire by moving the stopper to the second state when on the guidewire.
